(19) United States
(12) Patent Application Publication
(10) Pub. No.: US 2023/0053713 A1
(43) Pub. Date: Feb. 23, 2023

(54) DEVICES FOR TRANSRADIAL CATHETERIZATION

(71) Applicant: CRYSTALLINE MEDICAL, INC., Fremont, CA (US)

(72) Inventor: Theodore P. Abraham, San Francisco, CA (US)

(21) Appl. No.: 17/792,552

(22) PCT Filed: Jan. 5, 2021
(86) PCT No.: PCT/US2021/012132

§ 371 Date: Jul. 13, 2022

Related U.S. Application Data
(60) Provisional application No. 62/960,198, filed on Jan. 13, 2020.

(51) Int. Cl. A61F 5/01; A61F 5/37

(52) U.S. Cl. CPC A61F 5/013; A61F 5/3723

(57) ABSTRACT

The invention provides devices and methods for performing transradial catheterization.

### CROSS-REFERENCE TO RELATED APPLICATIONS

This application claims the benefit of, and priority to, U.S. Provisional Patent Application No. 62/960,198, filed Jan. 13, 2020, the contents of which are incorporated by reference.

### FIELD OF THE INVENTION

The invention relates generally to medical devices for catheterization.

### BACKGROUND

Heart disease is the leading killer in the United States. Percutaneous coronary intervention (PCI) is a non-surgical procedure used to treat stenosis of the coronary arteries in both urgent and chronic cardiovascular conditions. In patients with acute coronary syndromes, timely application of PCI is essential: it is estimate that 1% of brain function is lost for each minute that PCI is delayed.

Two routes are used to access to the coronary arteries by PCI, but each is problematic. Currently, the predominant route of access is via the femoral artery in the upper leg. One problem with transfemoral artery (TFA) access is the high rate of bleeding and access site complications associated with the procedure. In addition, TFA requires patients to lie flat during the procedure and tends to be uncomfortable. As a result, there is growing trend in favor of accessing the coronary arteries via the radial artery near the wrist. Transradial artery (TRA) access, however, is technically challenging due to the small size of arteries in the arm and the tendency of the arteries to spasm during catheterization. Moreover, patients must maintain their wrists in a hyperextended position throughout the procedure, which can be difficult. Consequently, there are significant barriers that curb the effectiveness of existing methods of PCI as well as serious side effects associated with such methods.

### SUMMARY

The invention provides devices and methods that allow safe, effective TRA catheterization to allow TRA-based coronary intervention. Among the devices are arm-securing devices that retain a patient's arm in a stable position with a hyperextended wrist and stabilization devices that couple the arm-securing devices to catheterization devices, included ultrasonic, image-guided catheters. The stabilization devices permit the catheterization device to be immobilized in relation to the patient's arm during the procedure to facilitate catheterization and minimize complications during the procedure. At the same time, the stabilization devices allows adjustment of the positioning of the catheterization device in relation to the arm-securing device so that the system can be adjusted to account for patient variables such as arm size, flexibility, and proximity of the radial artery to the skin. The invention further provides methods of using the aforementioned devices to perform transradial catheterization.

In an aspect, the invention provides devices that include an anchor configured to receive a catheterization device and a stabilizer coupled to the anchor and rotatable relative to the anchor.

The anchor may include an adjustable port for receiving the catheterization device. The catheterization device may have a longitudinal axis when it is coupled to the device, and the adjustable port may be adjustable along an axis perpendicular to the longitudinal axis of the catheterization device.

The stabilizer may be couplable to an arm-securing device configured to secure the arm of a human.

The device may include a catheterization device coupled to the anchor. The catheterization device may be reversibly coupled to the anchor.

The catheterization device may include an imaging device. The imaging device may be a forward-viewing ultrasound device. The ultrasound device may operate at a frequency of from about 1 MHz to about 200 MHz or from about 14 MHz to about 20 MHz. The ultrasound device may include a transducer that includes from about 20 to about 128 elements. The imaging device may have a flat face, a perpendicular face, or an angled face.

The catheterization device may be an image-guided catheter. Examples of image-guided catheters that may be used with any of the devices provided herein are described in U.S. Pat. Nos. 8,147,413; 8,147,414; 8,403,858; 8,403,859; 9,149,257; and 9,855,021, and in U.S. Patent Publication Nos. 2018/0116630; 2019/0142371; and 2019/0142373, the contents of each of which are incorporated herein by reference.

The catheter of the catheterization device may have a variable diameter. The diameter may be from about 2 mm to about 15 mm.

In another aspect, the invention provided devices for securing the arm of a human. The devices include a first portion for contacting a posterior surface of a forearm of an arm, a second portion for contacting a posterior surface of a hand of the arm, and a joint that connects the first portion to the second portion and permits the first portion to rotate relative to the second portion such that when the arm is secured to the device, a wrist of the arm is hyperextended.

The joint may include a locking mechanism that maintains the wrist in a hyperextended position when the arm is secured to the device. The joint may define an angle between a longitudinal axis of the first portion and a longitudinal axis of the second portion and permit rotation such that the angle may be from about 130° to about 180°. The joint may permit rotation in discrete increments. The increments may be from about 2° to about 5°.

The device may include one or more fasteners that secure the arm or portion of the arm to the device. The fastener may secure the forearm to the first portion of the device. The fastener may secure the hand to the second portion of the device. The fastener may be a strap, belt, or band. The fastener may include a hook-and-loop fastener system.

Each of the first and second portions of the device may be flat, curved, or concave. The surface of the first portion in contact with the posterior of the forearm may be flat, curved, or concave. The surface of the second portion in contact with the posterior of the hand may be flat, curved, or concave.

The device may include padding. The padding may be positioned on a surface of the first portion in contact with the posterior of the forearm. The padding may be positioned on a surface of the second portion in contact with the posterior of the hand.

The device may include a securing mechanism for securing a stabilization device to the arm-securing device. The securing mechanism may be a mount or band. The stabilization device may include an anchor configured to receive a catheterization device and a stabilizer coupled to the anchor and rotatable relative to the anchor. The stabilization device may include any of the elements described above in relation to devices containing an anchor and a stabilizer. The stabilization device may be coupled to a catheterization device. The catheterization device may include any of the elements described above.

In another aspect, the invention provides device for transradial catheterization. The devices include an arm-securing device configured to secure an arm of a human, a stabilization device coupled to the arm-securing device, and a catheterization device coupled to the stabilization device.

The longitudinal axis of the arm-securing device and the longitudinal axis of the catheterization device may lie in a plane. The arm-securing device and the catheterization device may be rotatable about an axis perpendicular to the plane.

The stabilization device may be releasably coupled to the arm-securing device. The arm-securing device may include one or more one securing mechanism that releasably receives the stabilization device. The securing mechanism may be a mount or band. The stabilization device may include an anchor configured to receive a catheterization device and a stabilizer coupled to the anchor and rotatable relative to the anchor.

The stabilization device may be releasably coupled to the catheterization device. The stabilization device may be adjustably coupled to the catheterization device such that the imaging device is movable along an axis perpendicular to the longitudinal axis of the arm-securing device. The stabilization device may be coupled to the catheterization device via a port on the stabilization device.

The arm-securing device may include any of the elements described above in relation to devices for securing the arm of a human. The arm-securing device may include a first portion for contacting a posterior surface of a forearm of the arm, a second portion for contacting a posterior surface of a hand of the arm, and a joint that connects the first portion to the second portion and permits the first portion to rotate relative to the second portion such that when the arm is secured to the device, a wrist of the arm is hyperextended. The arm-securing device may include a locking mechanism that maintains the wrist in a hyperextended position when the arm is secured to the device.

The catheterization device may include any of the elements described above. The catheterization device may include an imaging device.

In another aspect, the invention provides methods of transradial catheterization. The methods include securing the arm of a human to an arm-securing device coupled to a catheterization device and inserting a needle from the catheterization device into a radial artery in the arm.

The inserting step may include making a single puncture in the radial artery.

The arm may be secured in a position in which a wrist of the arm is hyperextended. The arm may be secured by contacting the posterior surface of the forearm to a first portion of the arm-securing device and contacting the posterior surface of the hand of the arm to a second portion of the arm-securing device.

The arm-securing device may be coupled to the catheterization device via a stabilization device. The stabilization device may include any of the elements described above in relation to devices containing an anchor and a stabilizer. The stabilization device may include an anchor secured to the catheterization device and a stabilizer coupled to the anchor and secured to the arm-securing device. The stabilization device may include any of the elements described above in relation to devices containing an anchor and a stabilizer.

The catheterization device may include any of the elements described above.

The method may include guiding a catheter from the catheterization device toward a heart of the human. The guiding step may include transmitting sound waves at a frequency of from about 1 MHz to about 200 MHz.

In another aspect, the invention provides kits for performing transradial catheterization. The kits may include one or more of the devices described above. The kit may include instructions for assembling the devices. The kit may include instructions for performing methods, such as any of the methods described above.

### BRIEF DESCRIPTION OF THE DRAWINGS

FIG. 1 is a side view of a schematic arm-securing device according to an embodiment of the invention.

FIG. 2 is an en face view of a schematic of an arm-securing device according to an embodiment of the invention.

FIG. 3 is a side view of a schematic of a stabilizer according to an embodiment of the invention.

FIG. 4 is an en face view of a schematic of a stabilizer according to an embodiment of the invention.

FIG. 5 is a side view of a schematic of a device that includes a catheterization device and an anchor according to an embodiment of the invention.

FIG. 6 is a schematic of a device for performing transradial catheterization according to an embodiment of the invention.

### DETAILED DESCRIPTION

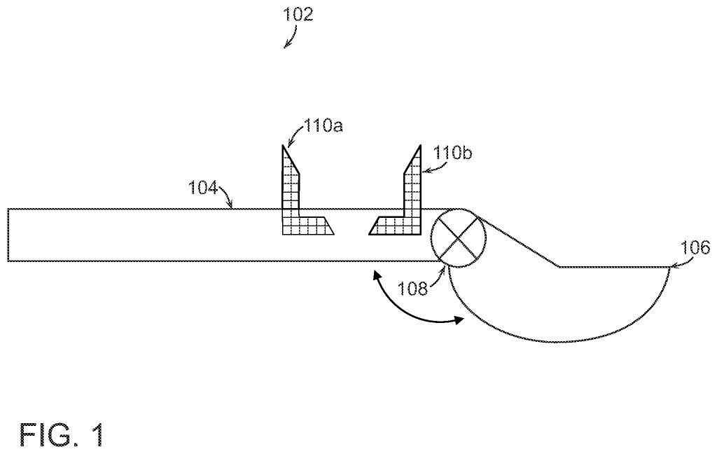

FIG. 1 is a side view of a schematic arm-securing device 102 according to an embodiment of the invention. The arm-securing device 102 includes a first portion 104 that accommodates the forearm and a second portion 106 that accommodates the hand. The posterior sides of the forearm and hand contact their respective portions of the arm-securing device 102. The first portion 104 and second portion 106 are connected by a joint 108 that permits the first portion 104 and second portion 106 to rotate relative to each other. The device includes securing mechanisms 110a and 110b that secure a stabilization device, described below, to the arm-securing device 102.

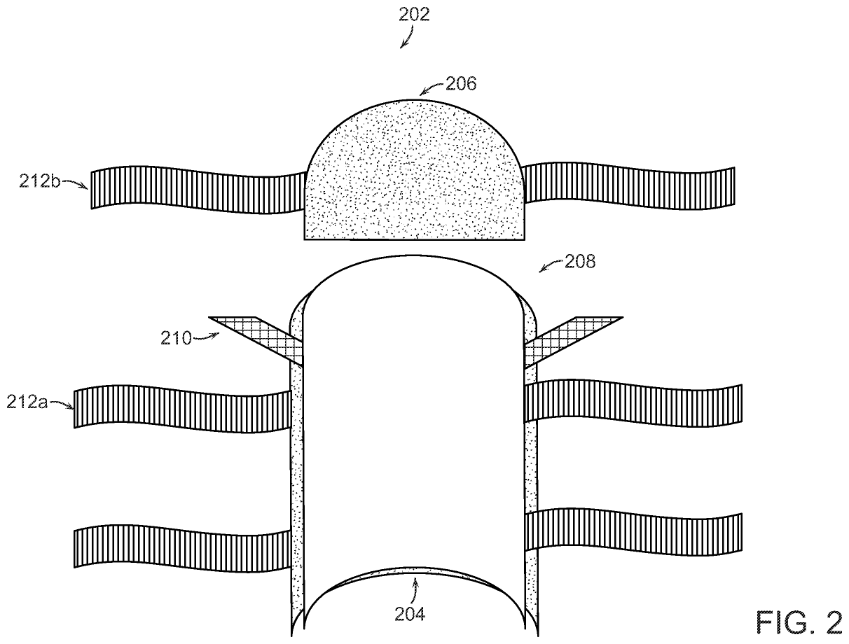

FIG. 2 is an en face view of a schematic of an arm-securing device 202 according to an embodiment of the invention. The arm-securing device 202 includes a first portion 204 that accommodates the forearm and a second portion 206 that accommodates the hand. The posterior sides of the forearm and hand contact their respective portions of the arm-securing device 202. The first portion 204 and second portion 206 are connected by a joint 208 that permits the first portion 204 and second portion 206 to rotate relative to each other. The device includes securing mechanism 210 that secure a stabilization device to the arm-securing device 202. The device also include fasteners 212a that secure the forearm to the first portion 204 and fasteners 212b that secure the hand to the second portion.

The joint permits rotation of the first and second portions relative to each other. In particular, when an arm is secured in the arm-securing device, the joint permit rotations of the hand backward from the forearm so that the wrist is hyperextended. The joint defines an angle between the longitudinal axis of the first portion and the longitudinal axis of the second portion. The joint may allow rotation so that the angle from about 130° to about 180°, with 180° being a straight line between the forearm and hand and 130° being the greatest degree of hyperextension of the wrist. The joint may include a locking mechanism that locks the joint a particular angle. The joint may be adjustable in discrete increments. For example and without limitation, the increments may be from about 2° to about 5°.

Each of the first and second portions of the arm-securing device may independently include padding on the surface contacting the posterior portion of the forearm or hand. The padding may include foam, fabric, or other cushioning material.

Any fastener suitable for securing the arm or portions of the arm to the arm-securing device may be used. For example and without limitation, the fasteners may be straps, belts, or bands. The fasteners may include a hook-and-loop fastener system, such as the one sold by the trade name Velcro by Velcro. Multiple types of fasteners may be used on in the arm-securing device. Each of the first and second portions may contain different types of fasteners or the same type of fasteners. The first and second portions may contain the same types of fasteners, or the two portions may contain different types of fasteners from each other.

The arm-securing device may include one or more securing mechanisms. The securing mechanisms may be attached to the first portion, the second portion or both. Any type of securing mechanisms may be used. For example and without limitation, the securing mechanisms may be mounts or bands.

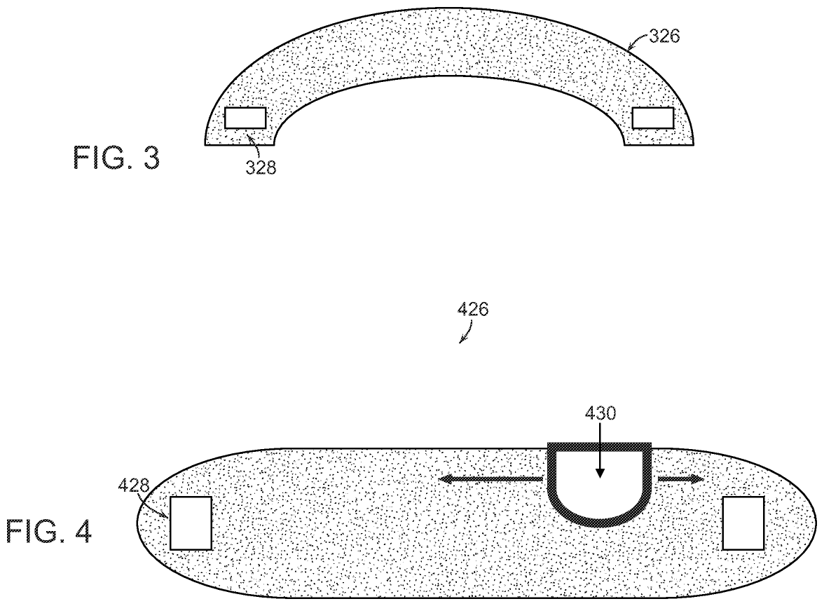

FIG. 3 is a side view of a schematic of a stabilizer 326 according to an embodiment of the invention. The stabilizer includes openings 328 that interact with securing mechanisms, such as mounts, on an arm-securing device.

FIG. 4 is an en face view of a schematic of a stabilizer 426 according to an embodiment of the invention. The stabilizer 426 includes openings 428 that interact with securing mechanisms, such as mounts, on an arm-securing device. The stabilizer 426 also includes a port 430 that for the anchor that secures a catheterization device, both of which are described below. The port is laterally slidable along the stabilizer to allow for adjustment of the position of the catheterization device.

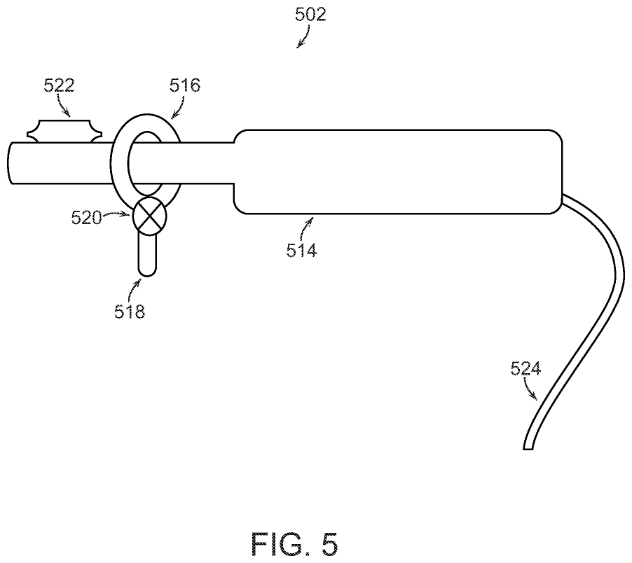

FIG. 5 is a side view of a schematic of a device 502 that includes a catheterization device 514 and an anchor according to an embodiment of the invention. The anchor includes a catheterization engagement portion 516, a lever 518, and a fulcrum 520. The catheterization engagement portion 516 engages the catheterization device 514 and secures it during use. The fulcrum 518 engages with the stabilizer and allows the anchor to rotate relative to the stabilizer while the device is in use. The catheterization device 514 includes a needle guide 522 that aligns the needle of the catheterization device 514 with an ultrasound beam. The catheterization device 514 includes a cable 524 to connect the catheterization device 514 to a power source, such as an outlet or battery.

The catheterization device may include an imaging device. The imaging device may be an ultrasound device, such as a forward-viewing ultrasound device.

The ultrasound device may operate at a frequency of from about 20 kHz to about 1000 MHz, from about 100 kHz to about 1000 MHz, from about 250 kHz to about 1000 MHz, from about 500 kHz to about 1000 MHz, from about 1 MHz to about 1000 MHz, from about 5 MHz to about 1000 MHz, from about 10 MHz to about 1000 MHz, from about 14 MHz to about 1000 MHz, from about 20 kHz to about 500 MHz, from about 100 kHz to about 500 MHz, from about 250 kHz to about 500 MHz, from about 500 kHz to about 500 MHz, from about 1 MHz to about 500 MHz, from about 5 MHz to about 500 MHz, from about 10 MHz to about 500 MHz, from about 14 MHz to about 500 MHz, from about 20 kHz to about 200 MHz, from about 100 kHz to about 200 MHz, from about 250 kHz to about 200 MHz, from about 500 kHz to about 200 MHz, from about 1 MHz to about 200 MHz, from about 5 MHz to about 200 MHz, from about 10 MHz to about 200 MHz, from about 14 MHz to about 200 MHz, from about 20 kHz to about 100 MHz, from about 100 kHz to about 100 MHz, from about 250 kHz to about 100 MHz, from about 500 kHz to about 100 MHz, from about 1 MHz to about 100 MHz, from about 5 MHz to about 100 MHz, from about 10 MHz to about 100 MHz, from about 14 MHz to about 100 MHz, from about 20 kHz to about 50 MHz, from about 100 kHz to about 50 MHz, from about 250 kHz to about 50 MHz, from about 500 kHz to about 50 MHz, from about 1 MHz to about 50 MHz, from about 5 MHz to about 50 MHz, from about 10 MHz to about 50 MHz, from about 14 MHz to about 50 MHz, from about 20 kHz to about 20 MHz, from about 100 kHz to about 20 MHz, from about 250 kHz to about 20 MHz, from about 500 kHz to about 20 MHz, from about 1 MHz to about 20 MHz, from about 5 MHz to about 20 MHz, from about 10 MHz to about 20 MHz, or from about 14 MHz to about 20 MHz,

The ultrasound device may include a transducer that includes from about 2 to about 1000 elements. from about 5 to about 1000 elements, from about 10 to about 1000 elements, from about 20 to about 1000 elements, from about 2 to about 500 elements. from about 5 to about 500 elements, from about 10 to about 500 elements, from about 20 to about 500 elements, from about 2 to about 250 elements. from about 5 to about 250 elements, from about 10 to about 250 elements, from about 20 to about 250 elements, from about 2 to about 128 elements. from about 5 to about 128 elements, from about 10 to about 128 elements, or from about 20 to about 128 elements.

The imaging device may have a flat face, a perpendicular face, or an angled face.

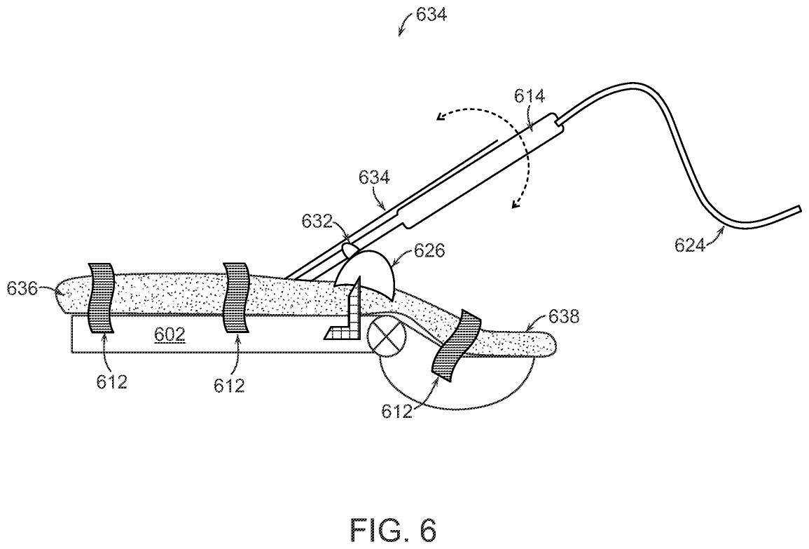

FIG. 6 is a schematic of a device 634 for performing transradial catheterization according to an embodiment of the invention. The device 634 includes a catheterization device 614 secured to an anchor 632. The catheterization device 614 includes a cable 624 for connecting to a power source and a needle 634 for puncturing the radial artery in the arm of a human. The catheterization device 614 is rotatable with respect to the anchor 632 as shown. The anchor 632 is coupled to a stabilizer 626, which in turn is secured to an arm-securing device 602. The arm-securing device 602 includes fasteners 612 that secure the forearm 636 and hand 638 of the patient's arm. Longitudinal axes of the catheterization device 614 and the arm-securing device 602 lie in a plane, and the catheterization device 614 is rotatable with respect to the stabilizer 626 about an axis perpendicular to that plane.

The invention also provides kits for performing transradial catheterization. The kits may include one or more of the devices described above. The kit may include instructions for assembling the devices or for performing transradial catheterization.

The inventor also provides methods of transradial catheterization. The methods include securing the arm of a human to an arm-securing device coupled to a catheterization device and inserting a needle from the catheterization device into a radial artery in the arm.

The inserting step may include making a single puncture in the radial artery.

The arm may be secured in a position in which a wrist of the arm is hyperextended. The arm may be secured by contacting the posterior surface of the forearm to a first portion of the arm-securing device and contacting the posterior surface of the hand of the arm to a second portion of the arm-securing device.

The arm-securing device may be coupled to the catheterization device via a stabilization device. The stabilization device may include any of the elements described above in relation to devices containing an anchor and a stabilizer. The stabilization device may include an anchor secured to the catheterization device and a stabilizer coupled to the anchor and secured to the arm-securing device. The stabilization device may include any of the elements described above in relation to devices containing an anchor and a stabilizer.

The catheterization device may include any of the elements described above.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this invention in its various embodiments and equivalents thereof.

1. A device comprising:
an anchor configured to receive a catheterization device; and
a stabilizer coupled to the anchor and rotatable relative to the anchor.

2. The device of claim 1, wherein the anchor comprises an adjustable port for receiving the catheterization device.

3. The device of claim 2, wherein:
the catheterization device comprises a longitudinal axis when the catheterization device is coupled to the device; and
the adjustable port is adjustable along an axis perpendicular to the longitudinal axis of the catheterization device.

4. The device of claim 1, wherein the stabilizer is couplable to an arm-securing device configured to secure an arm of a human.

5. The device of claim 1, further comprising the catheterization device coupled to the anchor.

6. The device of claim 5, wherein the catheterization device is reversibly coupled to the anchor.

7. The device of claim 5, wherein the catheterization device comprises an imaging device.

8. The device of claim 7, wherein the imaging device is a forward-viewing ultrasound device.

9. The device of claim 8, wherein the ultrasound device operates at a frequency of from about 1 MHz to about 200 MHz.

10. The device of claim 8, wherein the ultrasound device comprises a transducer comprising from about 20 to about 128 elements.

11. A device for securing an arm of a human, the device comprising:
a first portion for contacting a posterior surface of a forearm of an arm;
a second portion for contacting a posterior surface of a hand of the arm; and
a joint that connects the first portion to the second portion and permits the first portion to rotate relative to the second portion such that when the arm is secured to the device, a wrist of the arm is hyperextended.

12. The device of claim 11, wherein the joint comprises a locking mechanism that maintains the wrist in a hyperextended position when the arm is secured to the device.

13. The device of claim 12, wherein:
the joint defines an angle between a longitudinal axis of the first portion and a longitudinal axis of the second portion, and
the joint permits rotation such that the angle may be from about 130° to about 180°, with 130° corresponding to maximum hyperextension when the arm is secured to the device.

14. The device of claim 13, wherein the joint permits rotation in discrete increments of from about 2° to about 5°.

15. The device of claim 11, wherein:
the first portion comprises at least one first fastener for securing the forearm; and
the second portion comprises at least one second fastener for securing the hand.

16. The device of claim 15, wherein the at least one first fastener and the at least one second fastener are straps.

17. The device of claim 11, wherein a surface of the first portion that contacts the posterior surface of the forearm is concave.

18. The device of claim 11, wherein a surface of the first portion that contacts the posterior surface of the forearm is flat.

19. The device of claim 11, wherein the device comprises padding on at least one of:
a surface of the first portion, the surface of the first portion contacting the posterior surface of the forearm when the arm is secured to the device; and
a surface of the second portion, the surface of the second portion contacting the posterior surface of the hand when the arm is secured to the device.

20. The device of claim 11, wherein the first portion comprises at least one securing mechanism for securing a stabilization device coupled to a catheterization device.

21.-40. (canceled)
